(19) United States
(12) Patent Application Publication
(10) Pub. No.: US 2006/0105049 A1
(43) Pub. Date: May 18, 2006

(54) Folic acid-chitosan-DNA nanoparticles

(75) Inventors: Julio Fernandes, Brossard (CA); Francoise Winnik, Westmount (CA); Sania Mansouri, Montreal North (CA); Cuie Yan, Hubei (CN)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Valorisation Recherche HSCM & Universite de Montreal

(21) Appl. No.: 10/987,142

(22) Filed: Nov. 12, 2004

(51) Int. Cl. A61K 48/00; A61K 9/14; C08B 37/08

(52) U.S. Cl. 424/489; 514/44; 536/20; 977/916

(57) ABSTRACT

The present invention relates to a non-viral novel drug delivery system. Nanoparticles comprising folic acid and chitosan are used to deliver a therapeutic agent of interest to the cell for various therapeutic applications.

### BACKGROUND OF THE INVENTION

Gene therapy involves the introduction of exogenous genes into target cells for the purpose of achieving a therapeutic effect for the treatment of inherited and acquired diseases. Gene therapy relies on carriers such as viral or non-viral vectors for delivery. Viral gene delivery systems have been well characterized and include retroviruses, adenoviruses, adeno-associated viruses, herpes simplex virus and lentivirus (Oligino, T. J. et al., 2000, Clin. Orthop. 379 Suppl.: S17-30). Although these systems demonstrate high transfection efficiency when compared to non-viral vectors, they are accompanied by a number of drawbacks that severely hinder their use in vivo (Luo, D. et al., 2000, Nat. Biotechnol. 18: 33-37). Such limitations include their rapid clearance from the circulation, the reduced capacity to carry a large amount of genetic information and the associated risks of toxicity and immunogenic ity, which limits the possibility of subsequent administration. In addition, because of the random integration of some viral vectors into the genome, there is always a risk of insertional mutations which can contribute to the reactivation of tumors or other diseases.

The limitations of viral vectors, including the safety concerns surrounding their use, have led to an alternative approach based on non-viral systems. Naked DNA, cationic liposomes, cationic lipids and polymers, as well as DNA/cationic liposome/polycation complexes are utilized in the non-viral approach (Zelphati, O. et al., 1998, Gene Ther. 5: 1272-1282; Park, I. K. et al., 2001, J. Control. Rel. 76: 349-362; Gao, X. and Huang, L., 1996, Biochemistry 35: 1027-1036). The advantage of the non-viral method resides in the fact that it does not elicit an immune response and in its lack of toxicity for the cell (Romano et al., 2000, Stem Cells 18: 19-39). Moreover, non-viral systems can carry large therapeutic genes and can be produced in large quantities with high reproducibility at reduced production costs. For these reasons, there is an increased interest in the development of a safe and efficient non-viral gene delivery system that can circumvent the limitations encountered with the viral approach.

The active agent in a non-viral gene delivery system is the plasmid DNA. The vulnerability in vivo of naked plasmid DNA to enzymatic degradation, i.e. nucleases, has led to the investigation of complex systems to deliver the plasmid DNA. The complex formation between plasmid DNA and the carrier is initially electrostatic and results from the attraction between the anionic DNA and the cationic carrier. The aggregation of DNA with cationic lipids or polymers leads to a number of lipoplex or polyplex systems.

Indeed, the most commonly employed non-viral vectors are complexes composed of plasmid DNA and cationic lipids (Monck, M. A. et al., 2000, J. Drug Target 7: 439-452; Maurer et al., 1999, Mol. Membr. Biol. 16: 129-140). They are relatively large in size with positive charges that enhance their clearance from the circulation. Although they show an increased transfection efficacy in vitro, they have demonstrated toxicity both in vitro and in vivo (Li, S. and Huang, L., 1997, Gene Ther. 4: 891-900).

On the other hand, polymers offer some specific advantages over liposomes. The efficiency with which cationic polymers bind and condense plasmid DNA permits the protection of the nucleic acids during the intracellular transport (Dunlap et al., 1997, Nucl. Acids Res. 25: 3095-3101). Furthermore, through their biodegradation, polymers can ensure a controlled gene release which is a must for sustained protein expression. To achieve such versatility with a gene delivery system, various polymers have been studied, the first of which was poly(L-lysine) (Wu, G. Y. and Wu, C. H., 1987, J. Biol. Chem. 262: 4429-4432). Although it has been widely employed, it has demonstrated low transfection efficacy and evidence of cytotoxicity (Han, S. et al., 2000, Mol. Ther. 2: 302-317). In the case of poly(ethylenimine), it was shown that the level of transfection and cytotoxicity were closely related to the molecular weight of the polymer (Godbey, W. T. et al., 1999, J. Biomed. Mater. Res. 45: 268-275). For example, at a molecular weight of above 25 kDa, it displays high transfection efficiency, accompanied by cytotoxicity. Conversely, at lower molecular weights, there is negligible transfection with little toxicity. In addition to the previously mentioned polymers, others include poly(amidoamine), poly(D,L-lactic acid-co-glycolic acid) and chitosan to name a few (Han, S. et al., 2000, Mol. Ther. 2: 302-317).

Chitosan is a natural polycationic polysaccharide consisting of two subunits, D-glucosamine and N-acetyl-D-glucosamine, linked together by β(1,4) glycosidic bonds. Chitosan became an interesting biomaterial due to its low immunogenicity, minimal toxicity, good biocompatibility and biodegradability (Rao, S. B. et al., 1997, J. Biomed. Mater. Res. 34: 21-28; Richardson, S. C. et al., 1999, Int. J. Pharm. 178: 231-243). The cationic polyelectrolytic nature of the chitosan provides a strong electrostatic interaction with negatively charged DNA (Hejazi, R. and Amiji, M., 2003, J. Control. Rel. 89: 151-165; Fang, N. et al., 2001, Biomacromolecules 2: 1161-1168), and protects the latter from nuclease degradation in biological fluids (Cui, Z. and Mumper, R. J., 2001, J. Control. Rel. 75: 409-419; Illum, L. et al., 2001, Adv. Drug Deliv. Rev. 51: 81-96). These properties make chitosan a good candidate for the development of non-viral gene delivery system and/or drug delivery system (MacLaughlin, F. C. et al., 1998, J. Control. Rel. 56: 259-272; Richardson, S. C. W. et al., 1999, Int. J. Pharm. 178: 231-243). However, the transfection efficiency of chitosan-DNA nanoparticles is still very low in comparison with viral vectors.

It is an object of the present invention to provide a non-viral drug delivery system having an improved transfection efficiency.

### SUMMARY OF THE INVENTION

The present invention provides a nanoparticle made of a folic acid-chitosan conjugate. The nanoparticle comprises one or more therapeutic agents. In another embodiment, the invention relates to a drug delivery system for administration to a mammal comprising said nanoparticles.

The invention also provides a method of preparing the nanoparticles wherein folic acid and chitosan are reacted in solution and the resulting folic acid-chitosan conjugate is isolated. The folic acid-chitosan conjugate and the therapeutic agent are heated and mixed to form the nanoparticles of the invention.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a schematic representation of the folic acid-chitosan conjugation and resulting folic acid-chitosan polymer;

FIG. 2A is a graph representing the effect of charge ratio on size of folic acid-chitosan-DNA nanoparticles;

FIG. 2B is a graph representing the effect of charge ratio on zeta potential of folic acid-chitosan-DNA nanoparticles;

FIG. 3 is a graph plotting cell viability of HEK293 cells transfected with naked DNA, LipofectAMINE™2000, chitosan-DNA nanoparticles and folic acid-chitosan-DNA nanoparticles;

FIG. 4 is an agarose gel electrophoresis of chitosan-DNA and folic acid-chitosan-DNA nanoparticles digested with chitosanases and lysosymes to assess plasmid integrity.

FIG. 5 is a graph representing the transfection efficiency of chitosan-DNA nanoparticles incubated with HEK293 cells.

### DETAILED DESCRIPTION OF THE INVENTION

The invention relates to a novel non-viral drug delivery system. The Applicant has found that nanoparticles comprising folic acid in addition to chitosan show enhanced intracellular uptake of the non-viral vector. Folic acid is a natural receptor substrate present in the body. Its expression levels differ in healthy and diseased tissue. For example, folic acid receptors and non-epithelial isoform of folic acid receptors (FRβ) are consistently overexpressed, respectively, in various types of cancer cells including ovarian carcinoma, nasopharingeal carcinoma, cervical carcinoma, and choriocarcinoma (Antony, A. C., 1996, Ann. Rev. Nutr. 16: 501-521) and on activated synovial macrophages present in large numbers in arthritic joints in rheumatoid arthritis (Turk, M. J. et al., 2002, Arthritis Rheum. 46: 1947-1955). Consequently, the use of folic acid allows nanoparticle endocytosis via the folic acid receptor allowing for higher transfection yields. Additionally, the ability of folic acid to bind specifically to its receptor to allow endocytosis is not altered by covalent conjugation of small molecules (Wang, S. et al., 1997, Bioconj. Chem. 8: 673-679; Leamon, C. P. and Low, P. S., 1991, Proc. Natl. Acad. Sci. USA 88: 5572-5576; Lee, R. J. and Low, P. S., 1994, J. Biol. Chem. 4: 3198-3204).

The nanoparticles of the present invention are comprised of a folic acid-chitosan conjugate. The particles contain or encapsulate a suitable therapeutic agent which can include a DNA plasmid. It has been found that to ensure optimal uptake of the nanoparticles by the cells, the nanoparticles must have two properties: an appropriate surface charge or zeta potential and an appropriate size.

A positive surface charge allows an electrostatic interaction between negatively charged cellular membranes and positively charged nanoparticles. A positive zeta potential leads to a better interaction on the cellular membrane surface and allows for a more efficient uptake of the nanoparticles by the cells. The preferred zeta potential is in the range of between 3 mV and 20 mV. In a most preferred embodiment, the zeta potential is in the range of between 10 mV and 16 mV.

To increase endocytosis by cells, nanoparticle size should be between 50 nm and 500 nm. In a preferred embodiment, the nanoparticles have a size of between 50 to 200 nm. In a most preferred embodiment, nanoparticles having a size of less than 100 nm experience maximum endocytosis by non-specialized cells (Erbacher, P. et al., 1998, Pharm. Res. 15: 1332-1339).

In the preparation of the nanoparticles of the present invention, a folic acid-chitosan conjugate is first prepared. It is then dissolved by heating and mixed with the therapeutic agent which is to be delivered to the cell. The therapeutic agent includes but is not limited to a DNA plasmid containing one or more genes of interest, an oligonucleotide, a DNA sequence, a protein, a sequence or a drug inducing apoptosis, a biologically active molecule, a drug or other active agent.

In a preferred embodiment, the folic acid-chitosan conjugate is prepared by reacting a solution of 1-(3-dimethylaminopropyl)-3-ethylcarbodiimide hydrochloride and folic acid with chitosan as set out in Example 1.

### EXAMPLES

The invention is now described with reference to the following examples. These examples are provided for the purpose of illustration only and the invention should in no way be construed as being limited to these examples but rather should be construed to encompass any and all variations which become evident as a result of the teaching provided herein.

### Example 1

Synthesis of Folic Acid-Chitosan Conjugate.

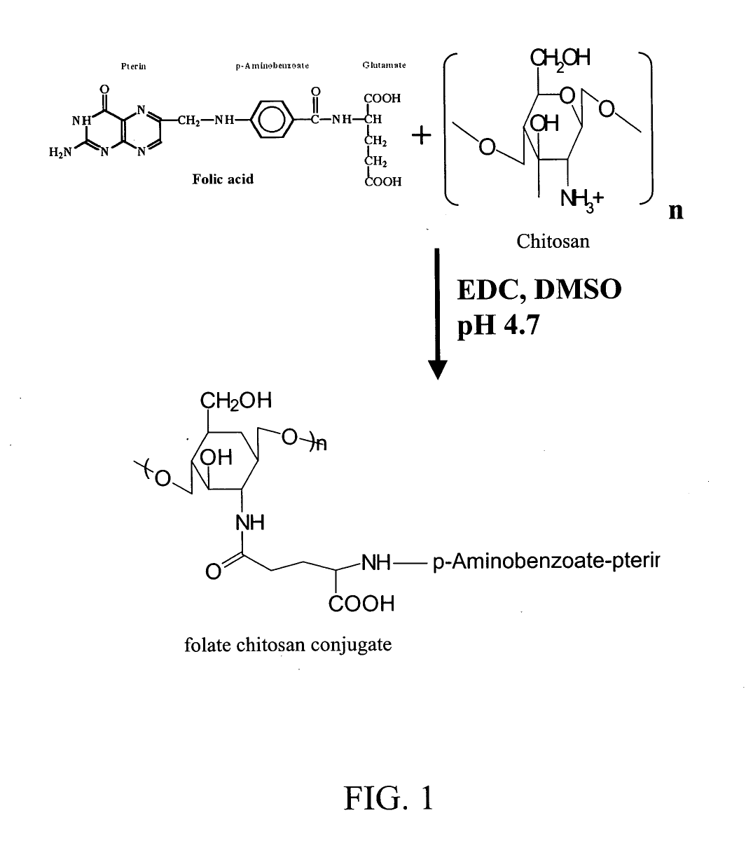

A solution of 500 mg of 1-(3-dimethylaminopropyl)-3-ethylcarbodiimide hydrochloride (EDC) and 500 mg of folic acid (Sigma-Aldrich, St. Louis, Mo., USA) in 12 ml of anhydrous dimethylsulfoxide (DMSO) (Sigma) is prepared and stirred for 1 hour at room temperature until the folic acid is dissolved. The folic acid preparation is added to a solution of 0.1% (w/v) chitosan (MW: 150 kDa, 85% degree of deacetylation obtained from Fluka Biokemica, Buchs, Switzerland) in acetate buffer (pH 4.7) and stirred, in the dark, for 16 hours at room temperature. The pH of the solution is brought to 9.0 by dropwise addition of diluted aqueous NaOH. The resulting mixture is dialyzed for a period of 3 days against phosphate buffer at pH 7.4 and 3 days against water. The resulting folic acid-chitosan polymer is then isolated by lyophilization. The reaction scheme and resulting polymer are illustrated in FIG. 1.

A preferred folic acid-chitosan conjugate has a molecular weight in a range between 5 to 600 kDa and a degree of deacetylation ranging between 70 to 95%. The preferred level of folic-acid conjugation ranges from 2 to 15 mol % folic acid per glucosamine residues.

The nanoparticles of the invention are then prepared by mixing the folic acid-chitosan conjugate or polymer with an appropriate therapeutic agent. It will be understood by a person skilled in the art that any suitable therapeutic agent may be used. The choice of therapeutic agent will depend on the therapeutic application sought. An example of the preparation of the nanoparticles is set out in Example 2 below where the therapeutic agent is in the form of a DNA plasmid.

### Example 2

1. Preparation of DNA Plasmid.

The VR1412 DNA plasmid (VICAL Inc., San Diego, Calif., USA) was purified using the Qiagen QIAfilter plasmid Giga kit (Mississauga, ON, Canada) according to the manufacturer's instructions and resuspended in water. The integrity of DNA plasmid was analyzed on a 0.8% agarose gel and DNA concentration was measured by UV absorbance at 260 nm (Corsi, K. et al., 2003, Biomaterials 24: 1255-1264).

2. Synthesis of Folic Acid-Chitosan-DNA Nanoparticles.

a) The folic acid-chitosan conjugate of Example 1 was dissolved in 20 mM acetic acid at pH 5.5 under low heating (inferior to 45° C.). The solution was then adjusted to a final concentration of 0.01% chitosan in 5 mM acetic acetate and sterile filtered through a 0.22 μm filter. The DNA plasmid solution was diluted in a 4.3 mM sodium sulfate solution to a concentration of 200 mg/ml. The folic acid-chitosan-DNA complex formation was achieved by a coacervation technique as described by Mao et al. (2001, J. Control. Rel. 70: 399-421) and Corsi et al. (2003, Biomaterials 24: 1255-1264). Folic acid-chitosan and DNA solutions were heated separately to 55° C. for 1 minute. Then, 950 μl of DNA solution was mixed with 50 μl of folic acid-chitosan and immediately vortexed at maximum speed for 1 minute. The final nanoparticle solution produced was used for the transfection experiments without further modification.

b) The folic acid-chitosan conjugate of Example 1 was dissolved in 20 mM acetic acid at pH 5.5 under low heating (inferior to 45° C.). The solution was then adjusted to a final concentration of 0.01% chitosan in 5 mM acetic acetate and sterile filtered through a 0.22 μm filter. The DNA plasmid solution was diluted in a 4.3 mM sodium sulfate solution to a concentration of 200 mg/ml. The folic acid-chitosan-DNA complex formation was achieved by a coacervation technique as described by Mao et al. (2001, J. Control. Rel. 70: 399-421) and Corsi et al. (2003, Biomaterials 24: 1255-1264). Folic acid-chitosan and DNA solutions were heated separately to 55° C. for 1 minute. Then, 900 μl of DNA solution was mixed with 100 μl of folic acid-chitosan and immediately vortexed at maximum speed for 1 min. The final nanoparticle solution produced was used for the transfection experiments without further modification.

c) The folic acid-chitosan conjugate of Example 1 was dissolved in 20 mM acetic acid at pH 5.5 under low heating (inferior to 45° C.). The solution was then adjusted to a final concentration of 0.01% chitosan in 5 mM acetic acetate and sterile filtered through a 0.22 μm filter. The DNA plasmid solution was diluted in a 4.3 mM sodium sulfate solution to a concentration of 200 mg/ml. The folic acid-chitosan-DNA complex formation was achieved by a coacervation technique as described by Mao et al. (2001, J. Control. Rel. 70: 399-421) and Corsi et al. (2003, Biomaterials 24: 1255-1264). Folic acid-chitosan and DNA solutions were heated separately to 55° C. for 1 minute. Then, 750 μl of DNA solution was mixed with 250 μl of folic acid-chitosan and immediately vortexed at maximum speed for 1 min. The final nanoparticle solution produced was used for the transfection experiments without further modification.

d) The folic acid-chitosan conjugate of Example 1 was dissolved in 20 mM acetic acid at pH 5.5 under low heating (inferior to 45° C.). The solution was then adjusted to a final concentration of 0.01% chitosan in 5 mM acetic acetate and sterile filtered through a 0.22 μm filter. The DNA plasmid solution was diluted in a 4.3 mM sodium sulfate solution to a concentration of 200 mg/ml. The folic acid-chitosan-DNA complex formation was achieved by a coacervation technique as described by Mao et al. (2001, J. Control. Rel. 70: 399-421) and Corsi et al. (2003, Biomaterials 24: 1255-1264). Folic acid-chitosan and DNA solutions were heated separately to 55° C. for 1 minute. Then, 500 μl of DNA solution was mixed with 500 μl of folic acid-chitosan and immediately vortexed at maximum speed for 1 min. The final nanoparticle solution produced was used for the transfection experiments without further modification.

e) The folic acid-chitosan conjugate of Example 1 was dissolved in 20 mM acetic acid at pH 5.5 under low heating (inferior to 45° C.). The solution was then adjusted to a final concentration of 0.01% chitosan in 5 mM acetic acetate and sterile filtered through a 0.22 μm filter. The DNA plasmid solution was diluted in a 4.3 mM sodium sulfate solution to a concentration of 200 mg/ml. The folic acid-chitosan-DNA complex formation was achieved by a coacervation technique as described by Mao et al. (2001, J. Control. Rel. 70: 399-421) and Corsi et al. (2003, Biomaterials 24: 1255-1264). Folic acid-chitosan and DNA solutions were heated separately to 55° C. for 1 minute. Then, 250 μl of DNA solution was mixed with 750 μl of folic acid-chitosan and immediately vortexed at maximum speed for 1 min. The final nanoparticle solution produced was used for the transfection experiments without further modification.

Laser light scattering and zeta potential measurements were used to assess folic acid-chitosan-DNA nanoparticle size and zeta potential versus the amino group to phosphate group ratio (N/P) as set out in Mao et al. (2001, J. Control. Release 70:399-421). This study was performed using a Malvern Zetasizer 4 (Malvern Inst. Ltd. Malvern, UK) as described in Mao et al. (2001, J. Control. Rel. 70: 399-421) and Erbacher et al. (1998, Pharm. Res. 15: 1332-1339). Solutions of different N/P ratios of chitosan-DNA and folic acid-chitosan-DNA nanoparticles were prepared to measure their size and their laser light scattering.

The size, zeta potential value and N/P ratio of the nanoparticles prepared in Examples 2 a) to 2 e) are summarized in Table 1.

TABLE 1
Size, zeta potential value and N/P ratio of nanoparticles a) to e).

| Nanoparticle | Size (nm) | Zeta potential value (mV) | N/P Ratio |
|---|---|---|---|
| a | 350 | −32 | 0.5 |
| b | 280 | +0.70 | 1 |
| c | 147 | +5.5 | 3.5 |
| d | 120 | +13.4 | 7 |
| e | 118 | +13.5 | 11 |

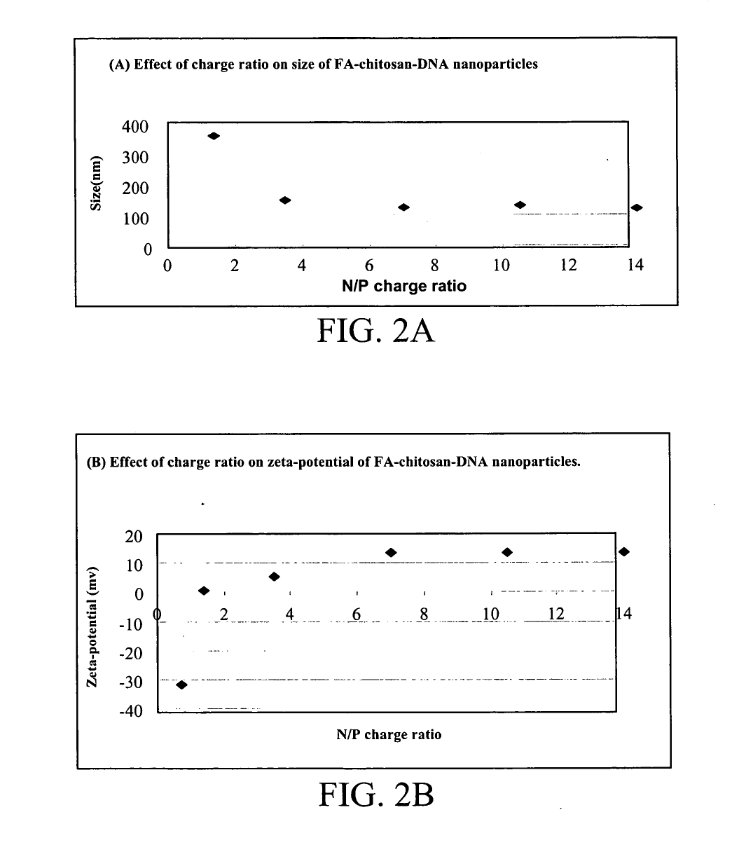

As shown in FIG. 2A, when the N/P ratio of folic acid-chitosan-DNA nanoparticles is about 1, the nanoparticle size is more than 300 nm. If the NIP ratio increases, the nanoparticle size decreases to 118 nm. In FIG. 2B, at a N/P ratio of 1 the zeta potential is 0 mV, but when the charge ratio increases beyond 7, the zeta potential levels off and remains stable at +15 mV. In a preferred embodiment, the N/P ratio is between 1 and 20. In a most preferred embodiment, the folic acid-chitosan-DNA nanoparticles of the invention have a preferred size of 118 nm and a N/P ratio of 7.

3. Cell Toxicity Assay.

For the nanoparticles of the present invention to have commercial applicability in the treatment of various diseases and the like, it must be determined that their use does not cause cell toxicity. As such, cell toxicity was assayed. To do so, human embryonic kidney 293 cells (HEK293) were obtained from the American Type Culture Collection (Manassas, Va., USA) and grown in minimal essential medium Eagle (MEM, obtained from Sigma-Aldrich, St-Louis, Mo., USA) at 37° C. in a 5-95% CO₂—O₂ atmosphere. Cells were seeded 24 hours prior transfection into a 24-well tissue culture plate at a density of 50,000 cells per well in 1 ml of MEM supplement with 10% FBS and 1% PS. At the time of transfection, the medium in each well was replaced with 500 μl of fresh complete medium containing 10 μg of folic acid-chitosan-DNA nanoparticles. Naked DNA and chitosan-DNA were used as controls. Following an overnight incubation, the cells received 1 ml of complete medium and incubated until 60 hours post-transfection LipofectAMINE™2000 (LF), a commercially available lipid vector, was used as a positive control according to the manufacturer's procedure. Each well of the tissue culture plate received 1 ml of LF that was complexed with 1 μg of DNA (Corsi, K. et al., 2003, Biomaterials 24: 1255-1264).

After 60 hours of incubation, the cytotoxicity of the complex FA-chitosan-DNA was determined by using 3-[4,5-dimethylthiazol-2-yl]-2,5-diphenyl tetrazolium bromide (MTT) assay. To each well of the tissue-culture plate, 100 μl of 5 mg/ml MTT was added. The samples were incubated for 4 hours at 37° C. Thereafter, the medium was removed and replaced with 200 μl of isopropanol—HCl (0.1N). The solutions were transferred to 96-well plates and the absorbance values were measured at 570 nm using an ELISA reader (Biotech, Fisher Scientific, Mississauga, ON, Canada). Viability of non-treated control cells was arbitrarily defined as 100% (Lee, K. Y. et al., 1998, J. Control. Rel. 51: 213-220; Corsi, K. et al., 2003, Biomaterials 24: 1255-1264; Rao, S. B. and Sharma, C. P., 1997, J. Biomed. Mater. Res. 34: 21-28).

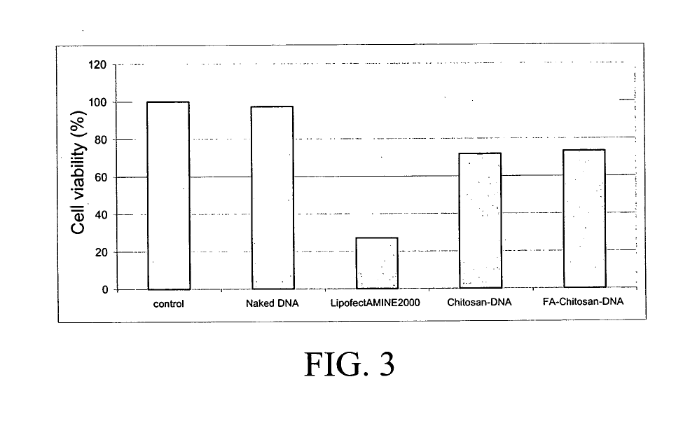

As shown in FIG. 3, when HEK293 cells were incubated with 10 μg of naked DNA, there was no significant change in cell viability compared to the control. A decrease in cell viability was observed when HEK293 cells were incubated with μg of chitosan-DNA and folic acid-chitosan-DNA, with no significant difference between the two types of conjugates. Finally, there was a significant decrease of cell viability when the HEK293 cells were incubated with DNA-LipofectAMINE™2000 lipoplex. This demonstrates that the folic acid-chitosan-DNA nanoparticles of the present invention do not impact negatively on cell viability.

4. Integrity of Therapeutic Agent within Nanoparticles.

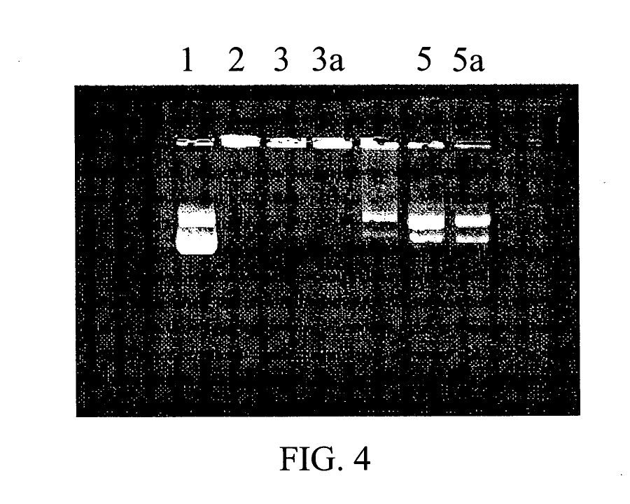

Plasmid DNA or any other therapeutic agent complexed with folic acid-chitosan must remain intact to ensure its functionality once inside the cell. Using an electrophoresis gel, the effect of synthesis conditions utilized and folic acid covalent binding with chitosan was assessed on DNA plasmid integrity. Naked DNA and nanoparticle suspensions subjected to chitosanase and lysosyme digestion as described in Mao et al. (2001, J. Control. Rel. 70: 399-421) and Corsi et al. (2003, Biomaterials 24: 1255-1264) were analyzed on a 0.8% agarose gel prepared in Tris-borate EDTA buffer pH 8.0 for 1 hour at 80 volts. The gel was stained with ethidium bromide (0.5 mg/ml) and rinsed with water before photography. The results presented in FIG. 4 demonstrate that the FA-chitosan conjugate protects the plasmid DNA against nuclease degradation (lanes 4, 5 and 5a), the band migration being comparable with the intact plasmid DNA before nanoparticle synthesis (lane 1). The plasmid DNA was released from the folic acid-chitosan conjugate following the digestion with chitosanase and lysozyme. The DNA presented in lanes 2, 3 and 3a was unable to migrate, indicating a strong attachment of the plasmid DNA to the chitosan (lane 2) and the folic acid-chitosan (lanes 3 and 3a). Moreover, in these lanes, there is no free DNA confirming the strong attachment with chitosan and folic acid-chitosan.

5. Transfection Efficiency.

In addition to protecting the plasmid DNA against nuclease degradation, an efficient delivery of the nanoparticle is required to transport the therapeutic gene or agent into the nucleus of the cell for its eventual release leading to gene expression and subsequent protein synthesis or therapeutic agent release. As such, the ability of chitosan-DNA nanoparticles to transfer in vitro a gene carrier, the β-gal gene, to HEK293 cells was determined. Chitosan was dissolved in 20 mM acetic acid at pH 5.5 under low heating (inferior to 45° C.). The solution was then adjusted to a final concentration of 0.01% chitosan in 5 mM acetic acetate and sterile filtered through a 0.22 μm filter. The DNA plasmid solution was diluted in a 4.3 mM sodium sulfate solution to a concentration of 200 mg/ml. The chitosan-DNA complex formation was achieved by a coacervation technique as described by Mao et al. (2001, J. Control. Rel. 70: 399-421) and Corsi et al. (2003, Biomaterials 24: 1255-1264). The chitosan and DNA solutions were heated separately for 1 minute to 55° C. for 1 minute. Then, 500 μl of DNA solution was mixed with 500 μl of chitosan and immediately vortexed at maximum speed for 1 min. The final nanoparticle solution produced was used for the transfection experiments without further modification.

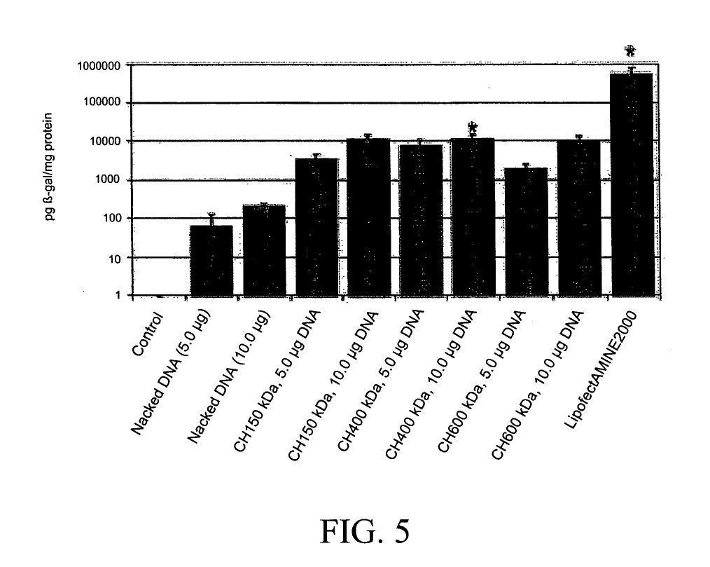

Cells were seeded 24 hours prior to transfection into a 24-well tissue culture plate at a density of 50,000 cells per well in 1 ml of their usual culture medium supplemented with 10% FBS and 1% PS. The day of transfection, the culture medium in each well was replaced with 500 μl of complete medium containing either naked DNA or chitosan-DNA nanoparticles having an amount of DNA equivalent to 5 to 10 μg. Following an overnight incubation, the cells received 1 ml of complete medium and were incubated until 60 hours post-transfection. LipofectAMINE™2000 was used as a positive control according to the manufacturer's procedures. To determine the transfection efficiency, the 13-gal expression was quantified using an ELISA kit according to the manufacturer's instructions. Briefly, 60 hours after transfection, the cells were lysed in a lysis buffer and centrifuged at maximal speed at 4° C. for 15 minutes to remove any debris. The β-galactosidase expression in the supernatant was determined as picogram of β-gal per milligram of cellular protein. Total protein content of the samples was measured using the BCA protein assay (Pierce, Rockford, Ill., USA). The results, presented in FIG. 5, are in accordance with those published by Mao et al. (2001, J. Control. Rel. 70: 399-421) and demonstrate that the chitosan-DNA nanoparticles entered the cell and led to the synthesis of the β-galactosidase protein. Moreover, the transfection efficiency is significantly higher when the cells were in contact with the chitosan (400 kDa)-DNA (10 μg) nanoparticles rather than the naked DNA.

As expression of the gene occurs with a chitosan-DNA nanoparticle, gene expression will be even higher when folic acid-chitosan-DNA nanoparticles are used since folic acid facilitates the internalization of the nanoparticles (Reddy et al., 2002, Gene Ther. 9: 1542-1550).

While the present invention has been described in connection with a specific embodiment thereof and in a specific use, various modifications will occur to those skilled in the art without departing from the spirit and scope of the invention as set forth in the appended claims. While the following claims are intended to recite the features of the invention, it will be apparent to those of skill in the art that certain changes may be made without departing from the scope of this invention.

What is claimed:

1. A nanoparticle made of a folic acid-chitosan conjugate, said nanoparticle comprising one or more therapeutic agents.

2. The nanoparticle of claim 1 having a mean size between 50 and 200 nm.

3. The nanoparticle of claim 1 having a N/P ratio of between 1 and 20.

4. The nanoparticle of claim 1 wherein the therapeutic agent is selected from the group consisting of a DNA plasmid, an oligonucleotide, a DNA sequence, a protein, a sequence inducing apoptosis, a drug inducing apoptosis, a biologically active molecule and a drug.

5. The nanoparticle of claim 4 wherein the therapeutic agent is a DNA plasmid.

6. The nanoparticle of claim 4 wherein the therapeutic agent is a sequence inducing apoptosis.

7. A drug delivery system for administration to a mammal comprising nanoparticles made of a folic acid-chitosan conjugate and comprising one or more therapeutic agents.

8. The drug delivery system of claim 7 wherein the nanoparticles have a mean size between 50 and 200 nm.

9. The drug delivery system of claim 7 wherein the nanoparticles have a N/P ratio of between 1 and 20.

10. The drug delivery system of claim 7 wherein the therapeutic agent is selected from the group consisting of a DNA plasmid, an oligonucleotide, a DNA sequence, a protein, a sequence inducing apoptosis, a drug inducing apoptosis, a biologically active molecule and a drug.

11. The drug delivery system of claim 10 wherein the therapeutic agent is a DNA plasmid.

12. The drug delivery system of claim 10 wherein the therapeutic agent is a sequence inducing apoptosis.

13. A method of delivering a therapeutic agent to a cell or tissue comprising the step of delivering nanoparticles to said cell or tissue, wherein said nanoparticles are made of a folic acid-chitosan conjugate and comprise one or more therapeutic agents.

14. The method of claim 13 wherein the nanoparticles have a mean size between 50 and 200 nm.

15. The method of claim 13 wherein the nanoparticles have a N/P charge ratio of between 1 and 20.

16. The method of claim 13 wherein the therapeutic agent is selected from the group consisting of a DNA plasmid, an oligonucleotide, a DNA sequence, a protein, a sequence inducing apoptosis, a drug inducing apoptosis, a biologically active molecule and a drug.

17. The method of claim 16 wherein the therapeutic agent is a DNA plasmid.

18. The method of claim 16 wherein the therapeutic agent is a sequence inducing apoptosis.

19. Use of the nanoparticle of any one of claims 1 to 6 to treat a condition or disease characterized by overexpression of the folic acid receptors on a cell surface.

20. Use of the nanoparticle of any one of claims 1 to 6 in the manufacture of a medicament to treat a condition or disease characterized by overexpression of the folic acid receptors on a cell surface.

21. Use of claim 19 or 20 wherein the condition or disease is cancer or rheumatoid arthritis.

22. A method of preparing a nanoparticle comprising the steps of:
a) reacting folic acid with chitosan in solution to obtain a folic acid-chitosan conjugate;
b) heating the folic acid-chitosan conjugate and heating a therapeutic agent; and
c) mixing the folic acid-chitosan conjugate and the therapeutic agent to obtain the nanoparticle.
